(19) United States
(12) Patent Application Publication
(10) Pub. No.: US 2025/0072836 A1
(43) Pub. Date: Mar. 6, 2025

(54) PULSE WAVE SIGNAL PROCESSOR, PHYSIOLOGICAL INFORMATION MEASUREMENT DEVICE, AND CONTACT-PRESSURE ABNORMALITY DETERMINATION METHOD

(71) Applicant: Murata Manufacturing Co., Ltd., Nagaokakyo-shi (JP)

(72) Inventor: Toru SHIMUTA, Nagaokakyo-shi (JP)

(21) Appl. No.: 18/950,638

(22) Filed: Nov. 18, 2024

Related U.S. Application Data
(63) Continuation of application No. PCT/JP2023/017001, filed on May 1, 2023.

(30) Foreign Application Priority Data
Jun. 1, 2022 (JP) 2022-089610

(51) Int. Cl. A61B 5/00; A61B 5/021

(52) U.S. Cl. CPC A61B 5/6843; A61B 5/6826; A61B 5/7239; A61B 5/7264; A61B 5/021; A61B 2560/0462; A61B 2562/0233

(57) ABSTRACT

A pulse wave feature calculator is provided that calculates a value of a pulse wave feature based on a steepness of a rise of a pulse wave measured with a photoplethysmographic sensor. Based on the value of the pulse wave feature calculated by the pulse wave feature calculator, a contact pressure determinator determines whether a contact pressure, which is a pressure with which the photoplethysmographic sensor is pressed against a measurement site, is within a range, such as an appropriate range.

### CROSS REFERENCE TO RELATED APPLICATIONS

This application is a continuation of International Application No. PCT/JP2023/017001, filed May 1, 2023, which claims priority to Japanese Patent Application No. 2022-089610, filed Jun. 1, 2022, the entire contents of each of which are hereby incorporated by reference in their entireties.

### TECHNICAL FIELD

The present disclosure relates to a pulse wave signal processor, a physiological information measurement device, and a contact-pressure abnormality determination method.

### BACKGROUND

Pulse waves that propagate in arteries are used as an index for estimating human health conditions. Pulse waves change with changes in blood flow volume at a measurement site. For compact wearable electronics, a convenient method for measuring pulse waves is to use a photoplethysmographic sensor. For example, light is emitted from a light-emitting element onto a measurement site such as a finger, and when the light is reflected from or transmitted through the measurement site, the light is detected by a light-receiving element to thereby acquire changes in blood flow volume as a pulse wave signal.

When the contact pressure between the photoplethysmographic sensor and the measurement site deviates from an appropriate range, the amplitude of a detected pulse wave decreases. For this reason, measuring a pulse wave with the photoplethysmographic sensor placed in contact with the measurement site can be performed with an appropriate contact pressure. For determination of whether the contact pressure is appropriate, wristwatch-type pulse wave signal processors incorporating a pressure sensor can be used, such as disclosed in International Publication No. 1994/15525. Such a pulse wave signal processor causes a display to display the current level of the contact pressure detected with the pressure sensor. If the value of the contact pressure is outside an appropriate range, the pulse wave signal processor can vibrate to notify the user to that effect.

### SUMMARY

Pulse wave signal processors in the related examples include an attached pressure sensor to determine whether the contact pressure is appropriate. Advances in miniaturization of pulse wave signal processors may make it difficult in some cases to attach such a pressure sensor. Further, the pressure sensor, and a component such as the light-emitting element or the light-receiving element are mounted at different locations. Consequently, even if the value of the contact pressure is appropriate at the location of the pressure sensor, this does not necessarily mean that the contact pressure is appropriate at the location where the component such as the light-emitting element or the light-receiving element is mounted.

Accordingly, it is an objects of the present disclosure to provide a pulse wave signal processor configured to determine, without use of a pressure sensor, whether the contact pressure of a component such as a light-emitting element or a light-receiving element against a measurement site is appropriate. It is another object of the present disclosure to provide a physiological information measurement device incorporating the pulse wave signal processor. It is still another object of the present disclosure to provide a contact-pressure abnormality method determination for determining, without use of a pressure sensor, whether the contact pressure is appropriate.

An exemplary aspect of the present disclosure provides a pulse wave signal processor. The pulse wave signal processor includes a pulse wave feature calculator configured to calculate a value of a pulse wave feature based on a steepness of a rise of a pulse wave measured with photoplethysmographic sensor; and a contact pressure determinator configured to, based on the value of the pulse wave feature, determine whether a contact pressure of the photoplethysmographic sensor pressing against a measurement side is within a range, such as an appropriate range.

Another exemplary aspect of the present disclosure provides a physiological information measurement device. The physiological information measurement device includes the pulse wave signal processor described above; and the photoplethysmographic sensor that makes a measurement of the pulse wave at the measurement site, and from which a result of the measurement is input to the pulse wave signal processor. For example, the photoplethysmographic sensor is configured to generate the pulse wave according to the measurement on the measurement site. The photoplethysmographic sensor includes at least one light-emitting element, and a light-receiving element that detects light, the light being output from the at least one light-emitting element and reflected from or transmitted through the measurement site.

Still another exemplary aspect of the present disclosure provides a contact-pressure abnormality determination method. The contact-pressure abnormality determination method includes calculating a pulse wave feature related to a steepness of rise of a pulse wave, the pulse wave being measured with a photoplethysmographic sensor in contact with a measurement site; and based on a value of the calculated pulse wave feature, determining whether a contact pressure is within an appropriate range, the contact pressure being a pressure with which the photoplethysmographic sensor is pressed against the measurement site.

The determination of whether the contact pressure is within an appropriate range is based on a pulse wave feature related to the steepness of rise of a pulse wave. This obviates the need to incorporate a pressure sensor that directly measures the contact pressure. This configuration in turn allows for miniaturization of a device that is to be brought into contact with a measurement site. Further, a pulse wave is used for the determination of whether the contact pressure is within an appropriate range. This configuration provides reliable physiological information to be calculated based on a pulse wave determined to be indicative of an appropriate contact pressure.

### BRIEF DESCRIPTION OF DRAWINGS

FIG. 1 illustrates, in block diagram, a pulse wave signal processor according to a first exemplary embodiment, and illustrates, in schematic form, a physiological information measurement device including the pulse wave signal processor.

FIG. 2 is a graph illustrating an example of a pulse wave, a velocity pulse wave, and an acceleration pulse wave.

FIG. 3 is a graph illustrating an example of a pulse wave and an acceleration pulse wave.

FIGS. 4A, 4B, 4C, and 4D are graphs each illustrating the relationship between the systolic blood pressure measured at the wrist, and the values of a pulse wave feature that are determined from pulse waves measured under the following conditions: when the height of a measurement site (finger) is varied relative to the heart: and when the elbow on the same side as the measurement site, which is the finger, is cooled with the measurement site positioned at the height of the chest.

FIG. 5 is a graph illustrating an example of changes with time in the amount of light that a light-receiving element receives when near-infrared light is output from a light-emitting element with a photoplethysmographic sensor placed in contact with the measurement site.

FIG. 6 is a graph illustrating the waveform of substantially one beat of pulse wave, when the contact pressure of the photoplethysmographic sensor against the measurement site is within an appropriate range and when the contact pressure is excessive.

FIG. 7 is a graph illustrating changes with time in the elapsed time from an a-wave peak to a b-wave peak in the pulse wave illustrated in FIG. 5.

FIG. 8 is a graph illustrating changes with time in a pulse wave feature “1/VE0.5” computed from the pulse wave illustrated in FIG. 5.

FIG. 9 is a graph illustrating changes with time in a pulse wave feature “a/S” computed from the pulse wave illustrated in FIG. 5.

FIG. 10 is a graph illustrating changes with time in a pulse wave feature “(a-b)/(a-d)” computed from the pulse wave illustrated in FIG. 5.

FIG. 11 is a flowchart illustrating steps involved in a contact-pressure abnormality determination method according to the first exemplary embodiment.

FIG. 12 illustrates, in block diagram, a pulse wave signal processor according to a second exemplary embodiment, and illustrates, in schematic form, a physiological information measurement device including the pulse wave signal processor.

FIG. 13 is a graph illustrating an example of changes with time in the amount of light that the light-receiving element receives when near-infrared light and green light are respectively output from two light-emitting elements with the photoplethysmographic sensor placed in contact with the measurement site.

FIG. 14 is a graph illustrating the waveform of substantially one beat of pulse wave measured with green light, when the contact pressure of the photoplethysmographic sensor against the measurement site is within an appropriate range and when the contact pressure is excessive.

FIG. 15 is a graph illustrating changes with time in a pulse wave feature “ab-time” computed from the pulse wave illustrated in FIG. 13.

FIG. 16 is a graph illustrating changes with time in the pulse wave feature “1/VE0.5” computed from the pulse wave illustrated in FIG. 13.

FIG. 17 is a graph illustrating changes with time in the pulse wave feature “a/S” computed from the pulse wave illustrated in FIG. 13.

FIG. 18 is a graph illustrating changes with time in the pulse wave feature “(a-b)/(a-d)” computed from the pulse wave illustrated in FIG. 13.

FIG. 19 is a graph illustrating the values of the pulse wave feature “ab-time” that are calculated based on pulse waves acquired from a plurality of subjects.

FIG. 20 is a graph illustrating the values of the pulse wave feature “1/(VE0.5) that are calculated based on pulse waves acquired from a plurality of subjects.

FIG. 21 is a graph illustrating the values of the pulse wave feature “a/S” that are calculated based on pulse waves acquired from a plurality of subjects.

FIG. 22 is a graph illustrating the values of the pulse wave feature “(a-b)/(a-d)” that are calculated based on pulse waves acquired from a plurality of subjects.

FIG. 23 illustrates, in block diagram, a pulse wave signal processor according to a modification of the second exemplary embodiment, and illustrates, in schematic form, a physiological information measurement device including the pulse wave signal processor.

FIG. 24 illustrates, in perspective view and in block diagram, a portion of a physiological information measurement device according to a third exemplary embodiment.

### DETAILED DESCRIPTION OF EXEMPLARY EMBODIMENTS

### First Exemplary Embodiment

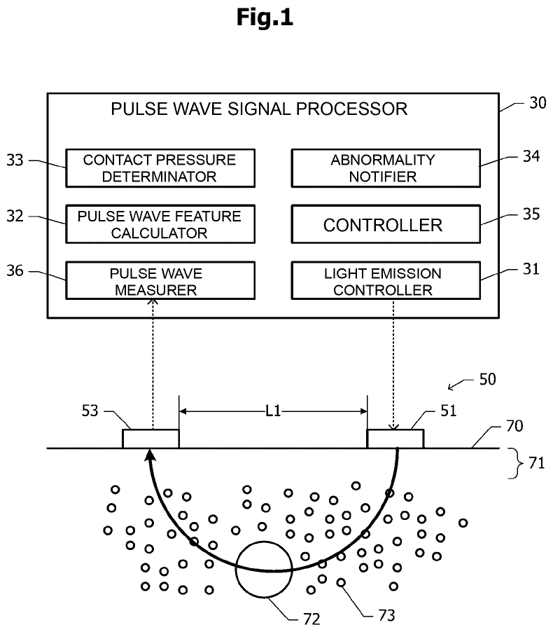

A pulse wave signal processor, a physiological information measurement device, and a contact-pressure abnormality determination method according to a first exemplary embodiment are described below with reference to FIGS. 1 to 11.

In general, it is known to those skilled in the art that pulse waves can be used for measurement of various physiological information. For example, pulse waves are used for purposes, such as measurement of pulse rate and measurement of oxygen saturation. Pulse waves are also used for purposes such as measurement of the autonomic function based on inter-pulse interval variations, measurement of respiration rate based on pulse wave baseline variations or inter-pulse interval variations. Further, techniques have been developed to estimate blood pressure from the waveform of a pulse wave. Pulse waves are classified into pressure pulse waves (e.g., piezoelectric pulse waves) measured with piezoelectric sensors or other sensors, and volume pulse waves (e.g., photoplethysmographic pulse waves) measured with photoplethysmographic sensors.

The pulse wave signal processor according to the first exemplary embodiment is applicable to both piezoelectric pulse waves and photoplethysmographic pulse waves. More information can be obtained from photoplethysmographic pulse waves than from piezoelectric pulse waves. The following description is directed to an example in which a photoplethysmographic pulse wave is used.

FIG. 1 illustrates, in block diagram, a pulse wave signal processor 30 according to the first exemplary embodiment, and illustrates, in schematic form, a physiological information measurement device including the pulse wave signal processor 30. The physiological information measurement device includes the pulse wave signal processor 30, and a photoplethysmographic sensor 50. The photoplethysmographic sensor 50 includes a light-emitting element 51, and a light-receiving element 53. The pulse wave signal processor 30 includes a light emission controller 31, a pulse wave feature calculator 32, a contact pressure determinator 33, an abnormality notifier 34, a controller 35, and a pulse wave measurer 36.

According to an exemplary aspect, the light-emitting element 51 and the light-receiving element 53 are used in contact with a body surface 70 of the user. The light-emitting element 51 applies measurement light, which is light used for measurement, onto the body surface 70. The applied light undergoes either absorption, or reflection or scattering (to be referred to simply as “reflection” hereinafter) by an epidermal region 71, arterioles 72, and capillaries 73 at the body surface 70. A portion of the reflected light is incident on the light-receiving element 53.

In general, the arterioles 72 are small blood vessels with a diameter of, for example, greater than or equal to 20 μm and less than or equal to 200 μm and lie between the arteries and the capillaries 73. The arterioles 72 each branch into a plurality of capillaries 73. The capillaries 73 are small blood vessels with a diameter of, for example, about 10 μm, and connects the arteries and the veins. The capillaries 73 are distributed in a region shallower than a region where the arterioles 72 are distributed. The arrow in FIG. 1 does not indicate the path of light propagation but indicates that light output from the light-emitting element 51 is incident on the light-receiving element 53 after passing through the epidermal region 71, the region where the capillaries 73 are distributed, and the region where the arterioles are distributed.

The light-emitting element 51 is configured to output measurement light under control by the light emission controller 31. The pulse wave feature calculator 32 is configured to receive input of a signal representing the intensity of light measured by the light-receiving element 53. For purposes of the exemplary aspects, a signal representing the intensity of light detected by the light-receiving element 53 is hereinafter referred to as “pulse wave signal.” Blood in the arteries contains hemoglobin. Hemoglobin has the property of absorbing the measurement light. Cardiac pulsation causes changes in blood flow volume, and such changes in blood flow volume also cause changes in the amount of light absorption. Accordingly, the intensity of a pulse wave signal changes with cardiac pulsation.

The light-emitting element 51 to be used outputs, for example, light within a range of wavelengths from blue to near-infrared (e.g., a range of wavelengths greater than or equal to 450 nm and less than or equal to 950 nm). Examples of the light-emitting element 51 to be used include a light-emitting diode (LED), and a vertical-cavity surface-emitting laser (VCSEL), for example. Examples of the light-receiving element 53 to be used include a photodiode (PD), and a phototransistor, for example.

Light with wavelengths shorter than 450 nm damages living tissues. At wavelengths longer than 950 nm, the absorbance of hemoglobin decreases. For acquisition of a pulse wave signal, it is thus desirable to use light within a range of wavelengths greater than or equal to 450 nm and less than or equal to 950 nm. Further, for detection of light within this wavelength range, a Si photodiode, which is inexpensive, can be used as the light-receiving element 53.

The controller 35 is configured to control, for example, the start and end of measurement, and display of the measurement results. The light emission controller 31 is configured to control the emission of pulsed light by the light-emitting element 51. For example, the light emission controller 31 causes the light-emitting element 51 to emit pulsed light at a predetermined frequency greater than or equal to 100 Hz and less than or equal to 1000 Hz.

The pulse wave measurer 36 is configured to generate a waveform of a pulse wave (to be sometimes referred to simply as “pulse wave”) from the measurement results (e.g., a pulse wave signal) input from the light-receiving element 53. For example, in synchronization with the emission of pulsed light by the light-emitting element 51, the pulse wave measurer 36 reads a measured value of light intensity from the light-receiving element 53 at a predetermined sampling rate to thereby generate a pulse wave.

The pulse wave feature calculator 32 calculates, from the pulse wave generated by the pulse wave measurer 36, a feature of the pulse wave (to be referred to as “pulse wave features” hereinafter). For example, the pulse wave is segmented on a beat-by-beat basis, and a pulse wave feature is determined from one beat of pulse wave.

Pulse wave features reflect physiological information such as pulse rate, oxygen saturation, respiration rate, blood pressure in large arteries, blood pressure in capillaries or arterioles (e.g., peripheral blood pressure), blood flow volume, vascular resistance, arterial stiffness, blood glucose levels, hemodynamics, and autonomic condition. Consequently, this physiological information can be estimated from pulse wave features determined by the pulse wave signal processor 30. Pulse wave features determined from a pulse wave actually measured with the photoplethysmographic sensor 50 are susceptible to the influence of the contact pressure with which the photoplethysmographic sensor 50 is pressed against a living body. This means that in estimating various physiological information from pulse wave features, if the contact pressure is not within an appropriate range, the reliability of the estimation results decreases.

The contact pressure determinator 33 is configured to determine, based on a pulse wave feature calculated by the pulse wave feature calculator 32, whether the contact pressure applied on the body surface 70 by the light-emitting element 51 and the light-receiving element 53 is appropriate. If the value of the contact pressure is outside an appropriate range (if the value of the contact pressure is abnormal), the abnormality notifier 34 notifies the user that the contact pressure is abnormal. For example, a notification of an abnormality in contact pressure is provided by means of sound, vibration, or light.

Reference is now made to pulse wave features that can be used for estimating physiological information.

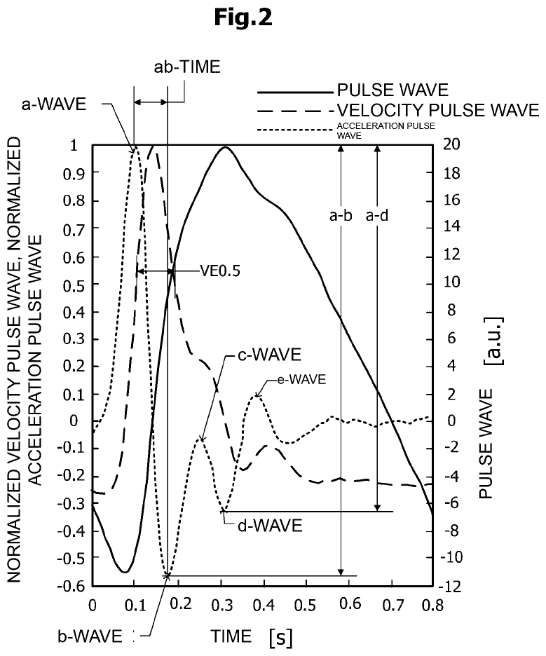

FIG. 2 is a graph illustrating an example of a pulse wave, a velocity pulse wave, and an acceleration pulse wave. The pulse wave feature calculator 32 is configured to calculate the first-order derivative and second-order derivative of a pulse wave. A waveform obtained from the first-order derivative of the pulse wave, and a waveform obtained from the second-order derivative of the pulse wave are respectively referred to as velocity pulse wave and acceleration pulse wave. For example, the intensity of the pulse wave, which is distributed discretely at time intervals corresponding to the sampling rate, is differentiated numerically at the time intervals corresponding to the sampling rate to thereby determine the velocity pulse wave. Further, the magnitude of the velocity pulse wave is differentiated numerically to thereby determine the acceleration pulse wave.

In FIG. 2, the horizontal axis represents time in units [s], the left vertical axis represents the respective magnitudes of a velocity pulse wave and an acceleration pulse wave, normalized to the maximum value of 1, and the right vertical axis represents the magnitude of the pulse wave in arbitrary units. In the graph of FIG. 2, the solid line, the long-dashed line, and the short-dashed line respectively represent the pulse wave, the velocity pulse wave, and the acceleration pulse wave. Generally, five peaks appear within each beat of acceleration pulse wave. The first, second, third, fourth, and fifth peaks within each beat of acceleration pulse wave are respectively referred to as a-wave, b-wave, c-wave, d-wave, and e-wave.

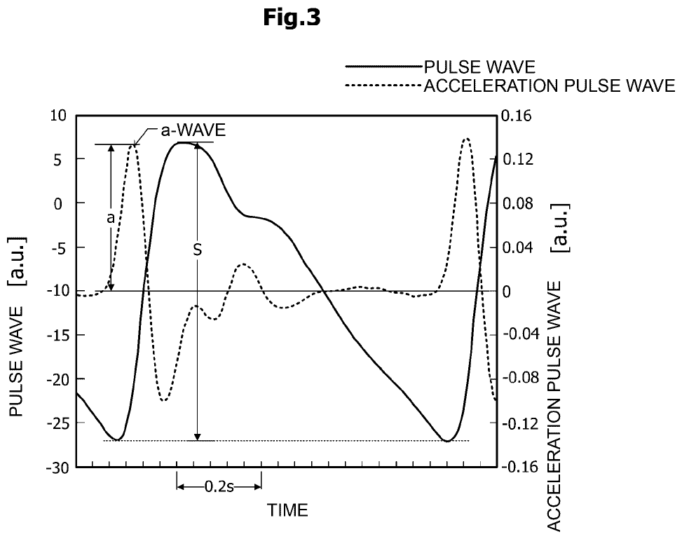

FIG. 3 is a graph illustrating an example of a pulse wave and an acceleration pulse wave. The horizontal axis represents time, the left vertical axis represents the magnitude of the pulse wave in arbitrary units, and the right vertical axis represents the magnitude of the acceleration pulse wave in arbitrary units. Five scale divisions on the horizontal axis correspond to 0.2 seconds. The ratio of the amplitude S of the pulse wave to the peak value “a” of the a-wave in the acceleration pulse wave is labeled “a/S.” The amplitude S of the pulse wave corresponds to the difference between the minimum value and the maximum value of the pulse wave after waveform correction is applied such that two consecutive pulse wave beats have the same minimum value.

Reference is now made to a method for estimating blood pressure from a pulse wave.

In general, blood pressure in the peripheral arterioles and capillaries is herein referred to as peripheral blood pressure. Although the term peripheral blood pressure is sometimes used to mean blood pressure at a site such as the wrist or ankle measured with a cuff-based sphygmomanometer, the blood pressure at a site, such as the wrist or ankle, is a measurement taken at a large artery (such as the radial artery), and in this sense differs from what is herein referred to as blood pressure measured at arterioles, capillaries, or other sites. The intravascular pressure decreases as blood travels from large arteries into arterioles and then capillaries. The degree of blood pressure decrease varies depending on factors such as the measurement site, the vascular state (such as the state of arteriosclerosis) of the user, the mental state (e.g., the autonomic state) of the user, environment (such as temperature or ambient noise), and the type of clothing worn.

Peripheral blood pressure is assumed to have two characteristics described below. First, under the condition of healthy blood vessels and no changes in vascular resistance, peripheral blood pressure has a positive correlation with the blood pressure measured at the upper arm, wrist, or other sites with a cuff-based sphygmomanometer. Second, when blood vessels are constricted upon cooling of an area near the measurement site, peripheral blood pressure decreases. An increase in vascular resistance due to constriction of peripheral blood vessels can, in some cases, cause an increase in blood pressure at the upper arm, wrist, or other sites.

Examples of pulse wave features that reflect the two characteristics mentioned above include the following three features: (1). the reciprocal of the full width at half maximum (the full width at half maximum being labeled “VE0.5” hereinafter) of the first upward peak in the velocity pulse wave (“1/(VE0.5)”); (2). the ratio of the amplitude S of the pulse wave to the peak value “a” of the a-wave in the acceleration pulse wave (to be labeled “a/S” hereinafter); and (3). the ratio between the difference (a-b), which is the difference between the a-wave peak value and the b-wave peak value in the acceleration pulse wave, and the difference (a-d), which is the difference between the a-wave peak value and the d-wave peak value (to be labeled “(a-b)/(a-d)” hereinafter).

Each of the pulse wave features mentioned above is herein referred to as “peripheral blood pressure index” According to an exemplary aspect.

As a pulse wave feature similar in characteristic to the peripheral blood pressure index, the following feature exists: the reciprocal of the time elapsed from the a-wave peak to the b-wave peak (to be labeled “1/(ab-time)” hereinafter).

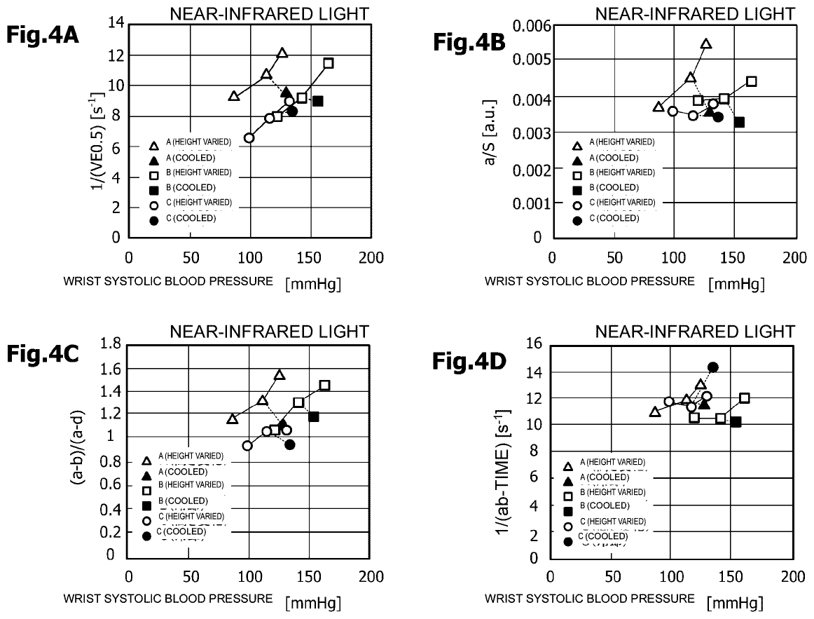

FIGS. 4A, 4B, 4C, and 4D are graphs each illustrating the relationship between the systolic blood pressure measured at the wrist, and the values of a pulse wave feature that are determined from pulse waves measured under the following conditions: when the height of a measurement site (e.g., a finger) is varied relative to the heart; and when the elbow on the same side as the measurement site, which is the finger, is cooled with the measurement site positioned at the height of the chest. Near-infrared light is used to measure the pulse waves. Near-infrared light penetrates into not only a shallow region where the capillaries 73 illustrated in FIG. 1 are distributed, but also a deep region where the arterioles 72 are distributed. Accordingly, the measured pulse waves reflect variations of blood flow in both the arterioles 72 and the capillaries 73.

Reference is now made to an exemplary range for the distance L (FIG. 1) from the light-receiving element 53 to the light-emitting element 51, when near-infrared light is used as measurement light. The distance L1 means the straight-line distance from the location where the light-emitting element 51 emits light to the location where the light-receiving element 53 receives light. As the distance L1 decreases, the amount of received light reflected from a shallow region immediately below the body surface 70 at the measurement site increases relative to the amount of received light reflected from a deeper region. This means that the influence of blood flow in the arterioles 72 is less likely to appear in the resulting pulse wave. Conversely, as the distance L1 increases, the amount of received light at the light-receiving element 53 decreases. To ensure that the influence of blood flow in the arterioles 72 be reflected in the pulse wave, and that a sufficient amount of light be received, the distance L1 is greater than or equal to 5 mm and less than or equal to 20 mm according to an exemplary aspect.

The horizontal axis in each of the graphs in FIGS. 4A to 4D represents systolic blood pressure at the wrist in units [mmHg]. The vertical axis in FIG. 4A represents the pulse wave feature “1/(VE0.5)” in units [s⁻¹]. The vertical axis in FIG. 4B represents the pulse wave feature “a/S” in arbitrary units. The vertical axis in FIG. 4C represents the pulse wave feature “(a-b)/(a-d)” in dimensionless form. The vertical axis in FIG. 4D represents the pulse wave feature “1/(ab-time)” in units [s⁻¹].

In each graph, the measurement results for three subjects, A, B, and C, are represented by triangle symbols, square symbols, and circle symbols, respectively. The three hollow symbols depicted for each subject represent the respective values of the corresponding pulse wave feature determined from pulse waves acquired with the measurement site (finger) set at the following heights: the abdomen (navel), the chest, and the face. The respective values of the pulse wave feature when the measurement site is set at the heights of the abdomen, the chest, and the face decrease in this order. The filled symbol depicted for each subject represents the value of the pulse wave feature determined from a pulse wave acquired while an area near the elbow is cooled with the measurement site set at the height of the chest.

It should be appreciated that although there are some differences between individual subjects, it can be observed that as the measurement site is varied in height, each of the four pulse wave features generally exhibits a positive correlation with the systolic blood pressure at the wrist.

With regard to each of the pulse wave features “1/(VE0.5)”, “a/S”, and “(a-b)/(a-d)”, the following tendency is observed: when the elbow is cooled, the pulse wave feature decreases in value, and the systolic blood pressure increases. The above-mentioned tendency of these pulse wave features is in agreement with the characteristics of the peripheral blood pressure mentioned above. It is to be noted that for some subjects, the pulse wave feature “1/(ab-time)” can increase when the elbow is cooled.

The pulse wave features “1/(VE0.5)”, “a/S”, “(a-b)/(a-d)”, and “1/(ab-time)” each depend on the steepness of rise of the pulse wave. These features are relatively insusceptible to the influence of the intensity of the pulse wave signal. In this regard, each of the values from which the pulse wave features “a/S” and “(a-b)/(a-d)” are calculated, that is, the a-wave peak value “a”, the b-wave peak value “b”, the d-wave peak value “d” of the acceleration pulse wave, and the amplitude S of the pulse wave, varies with the intensity of the pulse wave signal. However, taking the ratios of these values allows the influence of the intensity to be substantially eliminated.

Reference is now made to a method for determining whether the contact pressure is outside an appropriate range (e.g., an abnormal state).

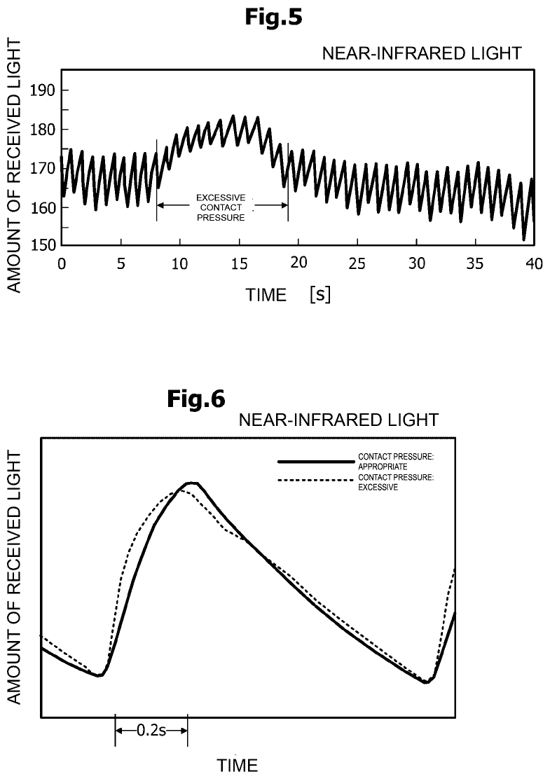

FIG. 5 is a graph illustrating an example of changes with time in the amount of light that the light-receiving element 53 receives when near-infrared light is output from the light-emitting element 51 with the photoplethysmographic sensor 50 (FIG. 1) placed in contact with the measurement site (photoplethysmographic pulse wave). The horizontal axis represents time in units [s], and the vertical axis represents the amount of received light in arbitrary scale divisions. In the observed pulse wave, the amount of received light increases and decreases on a beat-by-beat basis. Although the commonly known shape of the pulse wave is an upwardly projecting triangle, in FIG. 5, the shape is flipped vertically, and thus the pulse wave is depicted as having a waveform in the shape of a downwardly projecting triangle.

From 8 s to 19 s on the horizontal axis, the photoplethysmographic sensor 50 is pressed against the measurement site with an excessive contact pressure. It can be observed that application of excessive contact pressure causes an increase in the mean amount of received amount, and also causes a decrease in the amplitude of the pulse wave. The increase in the mean amount of received amount is due to the restriction of blood flow caused by the excessive contact pressure.

FIG. 6 is a graph illustrating the waveform of substantially one beat of pulse wave, when the contact pressure of the photoplethysmographic sensor 50 against the measurement site is within an appropriate range and when the contact pressure is excessive. The horizontal axis represents time, and the vertical axis represents the amount of received light. The solid line and the dashed line in the graph respectively represent the pulse wave for the case where the contact pressure is within an appropriate range, and the pulse wave for the case where the contact pressure is excessive. Waveform correction is applied to make the two pulse waves have substantially the same minimum value and substantially the same amplitude. It can be observed that when the contact pressure is excessive, the pulse wave rises more steeply than when the contact pressure is normal. As described above, an excessive contact pressure causes the waveform of the pulse wave to change. This means that the determination of whether the contact pressure is within an appropriate range wave can be made based on a change in the steepness of rise of the pulse wave.

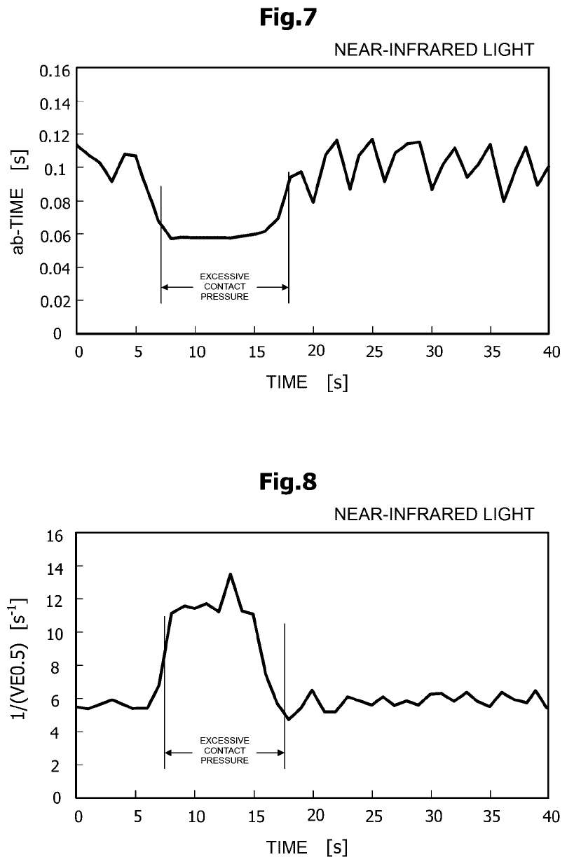

FIG. 7 is a graph illustrating changes with time in the elapsed time from the a-wave peak to the b-wave peak in the pulse wave (to be referred to as pulse wave feature “ab-time” hereinafter) illustrated in FIG. 5. The horizontal axis represents time in units [s], and the vertical axis represents the magnitude of the pulse wave feature “ab-time” in units [s]. It can be observed that the “ab-time” decreases during the period when the contact pressure is excessive. Detecting such a change in the “ab-time” can be used to determine whether the contact pressure is abnormal.

According to an exemplary aspect, the “ab-time” is used as a pulse wave feature for determining whether the contact pressure is abnormal. In one exemplary aspect, a determination threshold can be set in advance, and when the “ab-time” is less than or equal to the determination threshold, the contact pressure is determined to be abnormal. In another exemplary aspect, the reciprocal of the pulse wave feature “ab-time” can be used for the determination of whether the contact pressure is abnormal, and when the reciprocal of the “ab-time” is greater than or equal to the determination threshold, the contact pressure is determined to be abnormal.

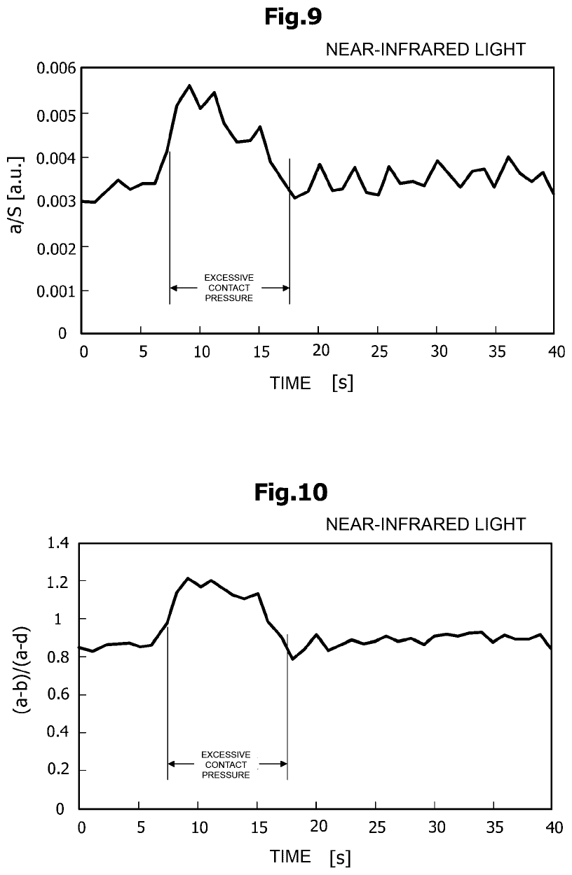

FIGS. 8, 9, and 10 are graphs respectively illustrating how the pulse wave features “1/(VE0. 5)”, “a/S”, and “(a-b)/(a-d)”, each of which is computed from the pulse wave illustrated in FIG. 5, change with time. The horizontal axis represents time in units [s]. The vertical axis in FIG. 8 represents the pulse wave feature “1/(VE0. 5)” in units [s⁻¹], the vertical axis in FIG. 9 represents the pulse wave feature “a/S” in arbitrary units, and the vertical axis in FIG. 10 represents the pulse wave feature “(a-b)/(a-d)” in dimensionless form. It can be observed that all of these pulse wave features increase in value during the period when the contact pressure is excessive, relative to the period when the contact pressure is appropriate.

These pulse wave features are related to the steepness of rise of the pulse wave. The reason why these features increase in magnitude during the period when the contact pressure is excessive is explained as follows.

When the contact pressure applied to the skin increases, the capillaries are compressed, and blood flow is impaired. As a result, blood accumulates in the arterioles located upstream of the capillaries. This causes an increase in blood pressure within the arterioles. If near-infrared light is to be used as measurement light, the influence of such increased blood pressure in the arterioles 72 is reflected significantly in the resulting pulse wave. This is considered to be the reason why these pulse wave features used as peripheral blood pressure indices increase in magnitude.

As described above, an excessive contact pressure causes the pulse wave features “1/(VE0.5)”, “a/S”, and “(a-b)/(a-d)” to increase in value. Accordingly, for example, a determination threshold is set in advance and, in response to these pulse wave features becoming greater than or equal to the determination threshold, the contact pressure is determined to be abnormal according to an exemplary aspect.

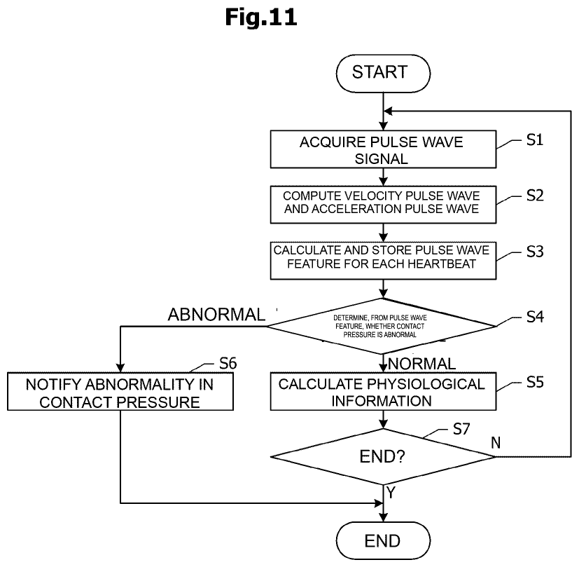

FIG. 11 is a flowchart illustrating steps involved in the contact-pressure abnormality determination method according to the first exemplary embodiment. The steps illustrated in FIG. 11 are to be executed by the pulse wave feature calculator 32 (FIG. 1).

First, the pulse wave feature calculator 32 (FIG. 1) acquires a pulse wave signal from the light-receiving element 53 (step S1). Subsequently, the first-order derivative of the pulse wave is taken to determine an acceleration pulse wave, and the second-order derivative of the pulse wave is taken to determine an acceleration pulse wave (step S2). Based on the pulse wave, the velocity pulse wave, and the acceleration pulse wave, a pulse wave feature is calculated for each heartbeat, and stored (step S3). For example, at least one of the following pulse wave features is calculated and stored: the pulse wave feature “ab-time” illustrated in FIG. 7; the pulse wave feature “1/(VE0.5)” illustrated in FIG. 8; the pulse wave feature “a/S” illustrated in FIG. 9; and the pulse wave feature “(a-b)/(a-d)” illustrated in FIG. 10.

Based on the calculated pulse wave feature, it is determined whether the contact pressure is abnormal (step S4). For example, the magnitude of the calculated pulse wave feature is compared with a determination threshold to determine whether the contact pressure is abnormal (step S4). The value of the pulse wave feature used at this time can be the mean, taken over a predetermined interval of time or a predetermined number of beats, of the values of the pulse wave feature that have been calculated for a plurality of individual heartbeats up to the current point in time.

If the value of the contact pressure is normal, various pieces of physiological information, for example, blood pressure, are determined based on the pulse wave, the velocity pulse wave, and the acceleration pulse wave (step S5). In another exemplary aspect, the respective peak times of waveforms are computed corresponding to individual beats, pulse rate is computed on a beat-by-beat basis by computing the inter-beat difference in peak time, and then the autonomic function is determined through the maximum entropy method or other methods from the time-series data of pulse rate. Steps S1 to S5 are repeated until the user performs an operation of ending the process (step S7). If the value of the contact pressure is abnormal, the abnormality notifier 34 (FIG. 1) is activated to notify the user of the abnormality (step S6), and the process is ended.

Reference is now made to advantages of the first exemplary embodiment.

According to the first exemplary embodiment, if the contact pressure is abnormal, the user is notified of the abnormality (step S6). This configuration allows the user to learn that the abnormality in the value of the contact pressure has resulted in decreased reliability of the computed values of various indices related to physiological information. This configuration further allows the user to adjust the contact pressure so as to make the contact pressure normal.

According to the first exemplary embodiment, information for determining an abnormality in contact pressure is acquired from the pulse wave. Consequently, the physiological information measurement device does not need to incorporate a pressure sensor that measures the contact pressure. This allows for decreased size and cost of the physiological information measurement device.

As another method for determining whether the contact pressure is abnormal without use of a pressure sensor, whether the contact pressure is abnormal can be also determined based on the absolute value of the amount of received light at the light-receiving element 53 (FIG. 1). The absolute value of the amount of received light, however, varies with the state of blood circulation at a measurement site. For example, poor blood circulation causes the amount of received light to increase. The absolute value of the amount of received light also varies with the measurement site. For example, the palmar side of the distal phalanx of the finger has a higher vascular density compared to sites such as the dorsal side of the finger, the middle phalanx, the proximal phalanx, the back of the hand, and the wrist. The absolute value of the amount of received light varies with the vascular density at the measurement site. Further, the absolute value of the amount of received light also varies with skin color. For example, with darker skin containing more melanin, the amount of received light decreases.

As described above, the absolute value of the amount of received light is affected by various factors other than the contact pressure. For this reason, using the method of determining an abnormality in contact pressure based on the absolute value of the amount of received light leads to decreased determination accuracy. According to the first exemplary embodiment, the absolute value of the amount of received light is not used for the determination of whether the contact pressure is normal. According to an exemplary aspect, high determination accuracy is maintained by eliminating the influence of various factors that affect the intensity of received light.

Reference is now made to modifications of the first exemplary embodiment.

According to the first exemplary embodiment, the determination of whether the contact pressure is abnormal is made by using at least one of the pulse wave features “ab-time”, “1/(VE0.5)”, “a/S”, and “(a-b)/(a-d)”, which are affected by the steepness of rise of the pulse wave. Alternatively, this determination can be made by using another pulse wave feature affected by the steepness of rise of the pulse wave. In this case, some exemplary examples use a pulse wave feature that is not likely to be affected by variation of the absolute value of the amount of received light at the light-receiving element 53.

According to the first exemplary embodiment, the value of a pulse wave feature measured with near-infrared light is used in determining whether the contact pressure is abnormal. Alternatively, other wavelengths of light with which the pulse wave feature changes in value upon application of excessive contact pressure can be used as measurement light. Other than near-infrared light, red light can be used. For example, light within a range of wavelengths greater than or equal to 600 nm and less than or equal to 950 nm can be used as measurement light.

According to the first exemplary embodiment, if it is determined at step S4 (FIG. 11) that the contact pressure is abnormal, then at step S6 (FIG. 11), the user is notified that the contact pressure is abnormal. Additionally, various physiological information calculated from a pulse wave, for example, pulse rate, oxygen saturation, autonomic function, respiration rate, blood pressure, vascular resistance, blood flow volume, peripheral blood pressure indices, hemodynamics, and blood glucose level can be displayed, and also a notification can be provided that indicates decreased reliability of these measured values due to an abnormal contact pressure. Some exemplary examples provide the user with a notification that instructs the user to decrease the contact pressure.

The user finds it annoying if the user receives such a notification frequently. Accordingly, under normal conditions, no such notification can be given, and when the state of abnormal contact pressure has continued for a long time, for example, for several hours or more, the user can be given the notification that the contact pressure is abnormal.

### Second Exemplary Embodiment

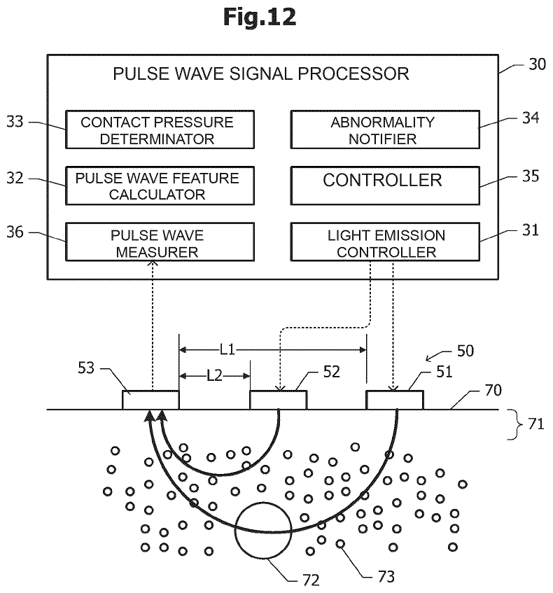

A pulse wave signal processor, a physiological information measurement device, and a contact-pressure abnormality determination method according to a second exemplary embodiment are now described below with reference to FIGS. 12 to 22. In the following, structural features similar to those of the pulse wave signal processor, the physiological information measurement device, and the contact-pressure abnormality determination method according to the first exemplary embodiment described above with reference to FIGS. 1 to 11 are not described in further detail.

FIG. 12 illustrates, in block diagram, the pulse wave signal processor 30 according to the second exemplary embodiment, and illustrates, in schematic form, a physiological information measurement device including the pulse wave signal processor 30. The photoplethysmographic sensor 50 of the physiological information measurement device according to the first exemplary embodiment (FIG. 1) includes a single light-emitting element 51 and a single light-receiving element 53. In contrast, the photoplethysmographic sensor 50 of the physiological information measurement device according to the second exemplary embodiment includes another light-emitting element 52 in addition to the light-emitting element 51.

The two light-emitting elements 51 and 52 output light with different wavelengths. For example, the light-emitting element 51 outputs near-infrared light with wavelengths greater than or equal to 850 nm and less than or equal to 950 nm, and the light-emitting element 52 outputs green light with wavelengths greater than or equal to 500 nm and less than or equal to 550 nm. The light-emitting element 52 to be used can be a light-emitting element that outputs light within a range of wavelengths from blue to yellow-green. The light-emitting element 52 is located closer to the light-receiving element 53 than is the light-emitting element 51. The distance from the light-receiving element 53 to the light-emitting element 52 is labeled L2. Near-infrared light undergoes less absorption in living bodies compared to green light. Near-infrared light thus penetrates into deeper regions of living bodies.

Accordingly, when near-infrared light is used as measurement light for measuring a pulse wave, changes in blood flow in the arterioles 72, which are distributed in a deep region, are reflected significantly in the pulse wave. When green light is used as measurement light for measuring a pulse wave, since green light does not penetrate into the deep region where the arterioles 72 are distributed, variations of blood flow in the capillaries 73, which are distributed in a shallow region, are reflected significantly in the pulse wave, whereas variations of blood flow in the arterioles 72 are less likely to be reflected in the pulse wave. The arrow in FIG. 12 that points from the light-emitting element 51 to the light-receiving element 53 indicates that information on blood flow in the arterioles 72 is reflected in the amount of received light at the light-receiving element 53. The arrow pointing from the light-emitting element 52 to the light-receiving element 53 indicates that information on blood flow in the capillaries 73 is reflected in the amount of received light at the light-receiving element 53, and that substantially no information on blood flow in the arterioles 72 is reflected in the amount of received light at the light-receiving element 53.

As described above with reference to the first exemplary embodiment, the distance L1 is greater than or equal to 5 mm and less than or equal to 20 mm according to an exemplary aspect. The distance L2 is greater than or equal to 1 mm and less than or equal to 3 mm to allow efficient acquisition of information about a region at a shallow depth from the body surface 70 according to an exemplary aspect.

The light emission controller 31 is configured to control the two light-emitting elements 51 and 52 to emit light at staggered timings. The pulse wave measurer 36 is configured to acquire a pulse wave measured with near-infrared light, and a pulse wave measured with green light separately by synchronizing, with the respective timings of light emission by the light-emitting elements 51 and 52, the timing at which a signal representing the intensity of received light is received from the light-receiving element 53.

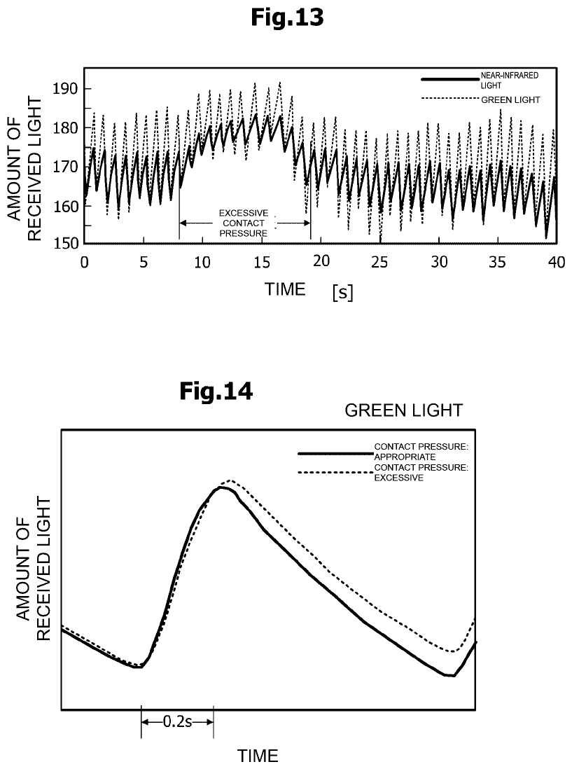

FIG. 13 is a graph illustrating an example of changes with time in the amount of light that the light-receiving element 53 receives when near-infrared light and green light are respectively output from the light-emitting elements 51 and 52 with the photoplethysmographic sensor 50 placed in contact with the measurement site. The horizontal axis represents time in units [s], and the vertical axis represents the amount of received light in arbitrary scale divisions. The thick solid line and the dashed line in the graph of FIG. 13 represent changes with time in the amount of received light (photoplethysmographic pulse wave), respectively for when near-infrared light is used as measurement light and for when green light is used as measurement light. The photoplethysmographic pulse wave obtained by use of near-infrared light as measurement light is identical to the photoplethysmographic pulse wave illustrated in FIG. 5.

It can be observed that in the period during which the photoplethysmographic sensor 50 is pressed against the measurement site with an excessive contact pressure, the pulse wave measured with green light exhibits, as with the pulse wave measured with near-infrared light, an increase in the mean amount of received light and also a decrease in amplitude. The increase in the mean amount of received amount is due to the restriction of blood flow caused by the excessive contact pressure.

FIG. 14 is a graph illustrating the waveform of substantially one beat of pulse wave measured with green light, when the contact pressure of the photoplethysmographic sensor 50 against the measurement site is within an appropriate range and when the contact pressure is excessive. The horizontal axis represents time, and the vertical axis represents the amount of received light. The solid line and the dashed line in the graph represent the waveform of the pulse wave, respectively, when the contact pressure is within an appropriate range and when the contact pressure is excessive. It can be observed that the pulse wave measured with green light exhibits a smaller change with variation of contact pressure than does the pulse wave measured with near-infrared light (FIG. 6). For example, the difference in the steepness of rise of the waveform between when the contact pressure is appropriate and when the contact pressure is excessive is small compared with that for the case where near-infrared light is used.

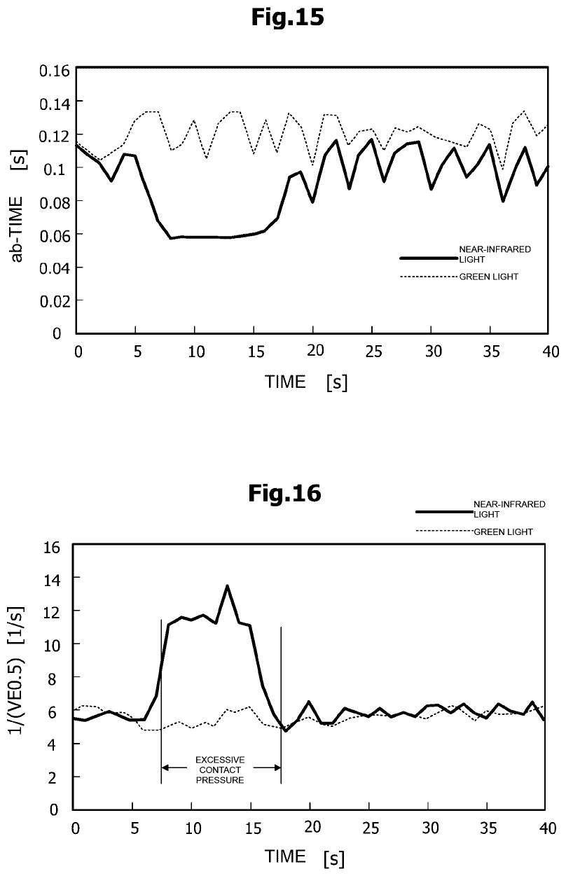

FIG. 15 is a graph illustrating changes with time in the pulse wave feature “ab-time” computed from the pulse wave illustrated in FIG. 13. The horizontal axis represents time in units [s], and the vertical axis represents the pulse wave feature “ab-time” in units [s]. The thick solid line and the dashed line in the graph of FIG. 15 respectively represent the “ab-time” computed based on the pulse wave measured with near-infrared light, and the “ab-time” computed based on the pulse wave measured with green light. Unlike the “ab-time” computed based on the pulse wave measured with near-infrared light, the “ab-time” computed based on the pulse wave measured with green light exhibits no noticeable change upon application of excessive contact pressure.

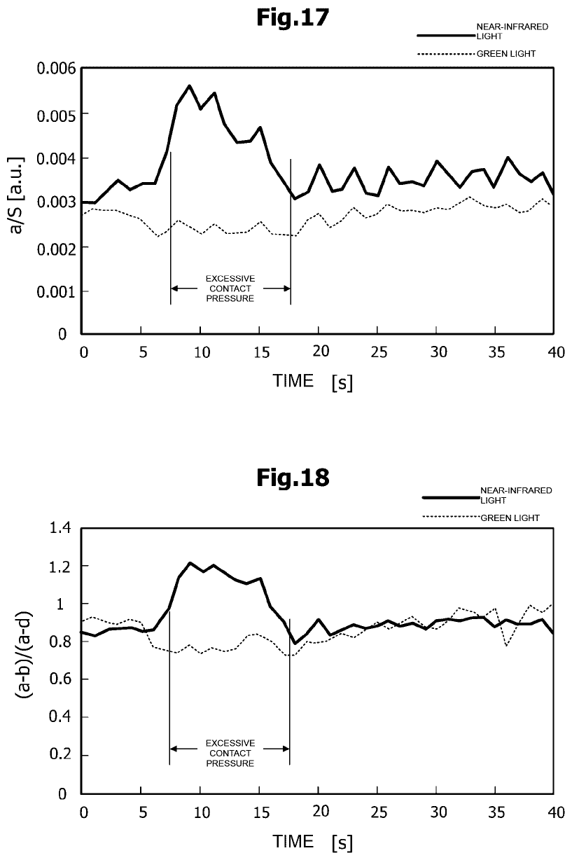

FIGS. 16, 17, and 18 are graphs respectively illustrating changes with time in the pulse wave features “1/(VE0.5)”, “a/S”, and “(a-b)/(a-d)” that are computed from the pulse wave illustrated in FIG. 13. The horizontal axis represents time in units [s]. The vertical axis in FIG. 16 represents the pulse wave feature “1/(VE0.5)” in units [s⁻¹], the vertical axis in FIG. 17 represents the pulse wave feature “a/S” in arbitrary units, and the vertical axis in FIG. 18 represents the pulse wave feature “(a-b)/(a-d)” in dimensionless form. The thick solid line and the dashed line in each of the graphs of FIGS. 16, 17, and 18 respectively represent the corresponding pulse wave feature computed based on the pulse wave measured with near-infrared light, and the corresponding pulse wave feature computed based on the pulse wave measured with green light. Of these pulse wave features, the pulse wave feature calculated based on the pulse wave measured with green light is observed to exhibit no noticeable change even upon application of excessive contact pressure, in comparison to the pulse wave feature calculated based on the pulse wave measured with near-infrared light. This is due to the fact that the change in the waveform of the pulse wave upon application of excessive contact pressure is small as illustrated in FIG. 14.

As described above, upon application of excessive contact pressure, a significant change is observed in the pulse wave feature calculated based on the pulse wave measured with near-infrared light, whereas no noticeable change is observed in the pulse wave feature calculated based on the pulse wave measured with green light. The reason for this can be explained as follows.

When the contact pressure applied to the skin increases, the capillaries 73 (FIG. 12) are compressed, and blood flow is impaired. As a result, blood accumulates in the arterioles 72 (FIG. 12) located upstream of the capillaries 73. This causes an increase in blood pressure within the arterioles 72. When near-infrared light is used as measurement light for measuring a pulse wave, the increase in blood pressure within the arterioles 72 is reflected in the pulse wave, and this is considered to be the reason for the increased value of the corresponding pulse wave feature.

When green light is used as measurement light for measuring a pulse wave, the increase in blood pressure within the arterioles 72 is hardly reflected in the pulse wave, and changes in blood pressure within the capillaries 73 are mainly reflected in the pulse wave. Compression of the capillaries 73 upon application of excessive contact pressure causes an increase in vascular resistance and a decrease in blood flow volume. Since blood pressure depends on the product of blood flow volume and vascular resistance, no noticeable change is observed in blood pressure within the capillaries 73. This is considered to be the reason why no noticeable change in pulse wave feature is observed when green light is used as measurement light.

As illustrated in each of FIGS. 15, 16, 17, and 18, when the contact pressure becomes excessive, the pulse wave feature measured with near-infrared light changes significantly, whereas no noticeable change is observed for the pulse wave feature measured with green light. In other words, when the contact pressure becomes excessive, the difference between the pulse wave feature measured with near-infrared light and the pulse wave feature measured with green light increases in comparison to when the contact pressure is within an appropriate range. The contact pressure determinator 33 (FIG. 12) makes a comparison between the pulse wave feature measured with near-infrared light and the pulse wave feature measured with green light, and based on the result of the comparison between the two pulse wave features, determines whether the contact pressure is abnormal.

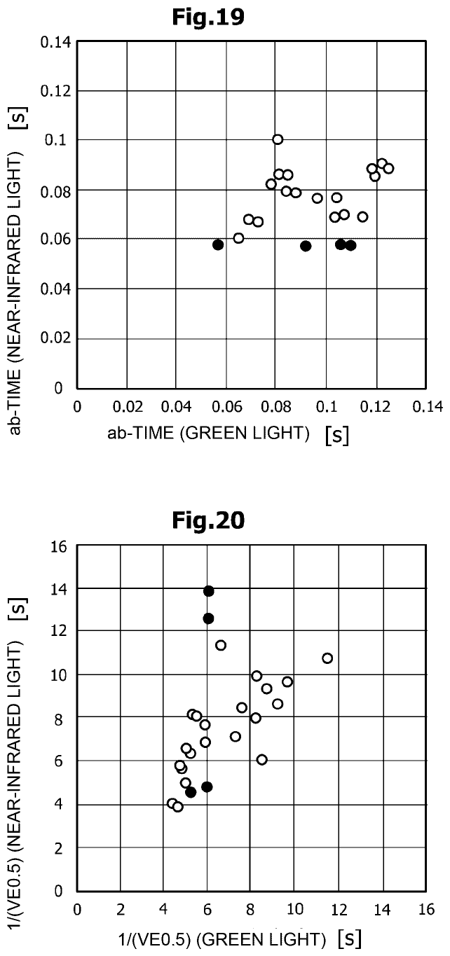

FIGS. 19, 20, 21, and 22 are graphs respectively illustrating the pulse wave features “ab-time”, “1/(VE0. 5)”, “a/S”, and “(a-b)/(a-d)” that are calculated based on pulse waves acquired from a plurality of subjects. In each graph, the horizontal axis represents a pulse wave feature calculated based on a pulse wave measured with green light, and the vertical axis represents a pulse wave feature calculated based on a pulse wave measured with near-infrared light. The hollow circle symbols each represent the pulse wave feature based on a pulse wave measured when the contact pressure is within an appropriate range, and the filled circle symbols each represent the pulse wave feature based on a pulse wave measured when the contact pressure is excessive.

It can be observed from FIG. 19 that there are subjects for whom the respective values of the pulse wave feature “ab-time” measured with near-infrared light and measured with green light are substantially equal, whereas there are also many subjects for whom the pulse wave feature “ab-time” measured with near-infrared light is shorter than the pulse wave feature “ab-time” measured with green light. As for the case where the contact pressure is excessive, it can be observed that the pulse wave feature “ab-time” measured with near-infrared light is less than or equal to 0.06 s for all of the subjects for whom the contact pressure has been confirmed to be excessive. It can therefore be said that the pulse wave feature “ab-time” measured with near-infrared light is not dependent on individual subjects and is thus an index for determining whether the contact pressure is abnormal according to an exemplary aspect.

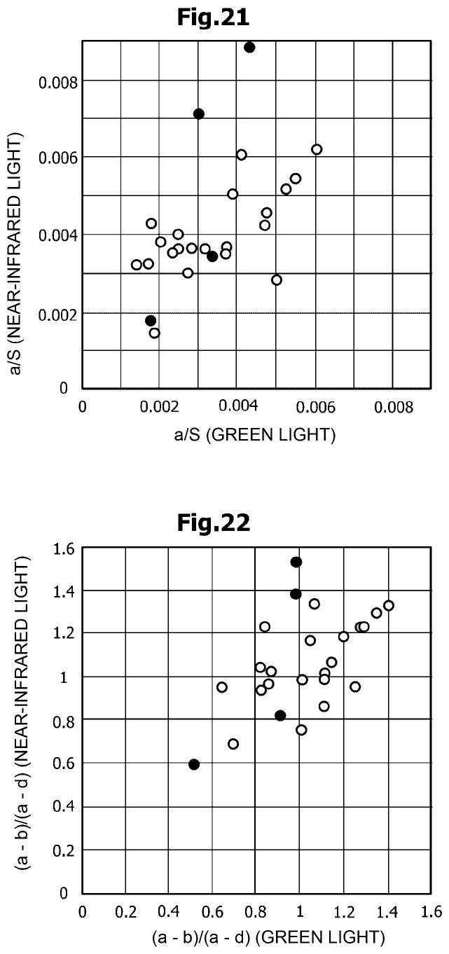

It can be observed from FIG. 20 that there are subjects for whom the respective values of the pulse wave feature “1/(VE0.5)” measured with near-infrared light and measured with green light are substantially equal, whereas there are also many subjects for whom the pulse wave feature “1/(VE0.5)” measured with near-infrared light is greater in value than the pulse wave feature “1/(VE0.5)” measured with green light. For the case where the contact pressure is abnormal, there are subjects for whom the pulse wave feature “1/(VE0.5)” measured with near-infrared light is greater in value than the pulse wave feature “1/(VE0.5)” measured with green light, whereas there are also subjects for whom the two pulse wave features are observed to be substantially equal.

The distribution of the values of the pulse wave features “a/S” illustrated in FIG. 21, and the distribution of the values of the pulse wave features “(a-b)/(a-d)” illustrated in FIG. 22 are both observed to be similar to the distribution of the values of the pulse wave features “1/(VE0.5)” illustrated in FIG. 19. The distributions of the values of the features illustrated in FIGS. 19, 20, and 22 can be explained as follows.

Upon application of excessive contact pressure, the capillaries 73 are compressed, and blood flow is impaired. As a result, blood accumulates in the arterioles 72 located upstream of the capillaries 73. Due to the resulting increase in blood pressure within the arterioles 72, the pulse wave features “1/(VE0.5)”, “a/S”, and “(a-b)/(a-d)” measured with near-infrared light increase in value. At this time, no noticeable change is observed for each of these pulse wave features measured with green light. Those subjects for whom, upon application of excessive contact pressure, the pulse wave feature measured with near-infrared light becomes greater in value than the pulse wave feature measured with green light are considered to be in this state.

Upon application of further excessive force, the arterioles 72 are also compressed, and blood flow is impaired. This results in a decreased difference between the pulse wave feature measured with near-infrared light, and the corresponding pulse wave feature measured with green light. Those subjects for whom, upon application of excessive contact pressure, the pulse wave feature measured with near-infrared light and the pulse wave feature measured with green light become substantially equal in magnitude are considered to be in this state.

It can be observed from the foregoing discussion that the pulse wave features “1/(VE0. 5)”, “a/S”, and “(a-b)/(a-d)” can be effectively utilized to determine whether the contact pressure is excessive within a certain range.

In particular, as illustrated in FIG. 19, the pulse wave feature “ab-time” measured with near-infrared light decreases for all subjects when the contact pressure becomes excessive, relative to when the contact pressure is within an appropriate range. Accordingly, a given determination threshold is set in some exemplary examples, and, in response to a measured value of the pulse wave feature “ab-time” becoming less than or equal to the determination threshold, the contact pressure is excessive can be determined. Alternatively, in some exemplary aspects, an estimation equation is created in advance such that the degree of excessiveness of the contact pressure increases with decreasing pulse wave feature “ab-time”, and the degree of excessiveness of the contact pressure is estimated from a measured value of the pulse wave feature “ab-time.”

Reference is now made to advantages of the second exemplary embodiment.

According to the second exemplary embodiment, the value of a pulse wave feature measured with near-infrared light, and the value of a pulse wave feature measured with green light are compared with each other to determine whether the contact pressure is abnormal. This allows for increased determination accuracy.

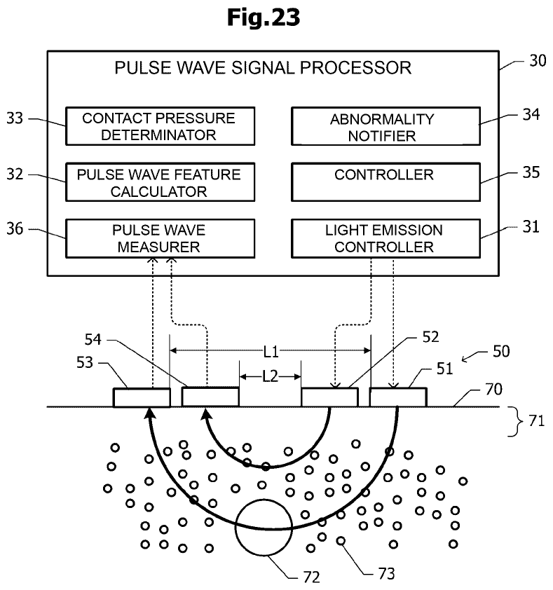

Reference is now made to FIG. 23 to describe a physiological information measurement device according to a modification of the second exemplary embodiment.

FIG. 23 illustrates, in block diagram, the pulse wave signal processor according to the modification of the second exemplary embodiment, and illustrates, in schematic form, a physiological information measurement device including the pulse wave signal processor. According to the second exemplary embodiment (FIG. 12), the two light-emitting elements 51 and 52 emit light at different wavelengths, and light emitted from each light-emitting element and reflected from the measurement site is detected by a single light-receiving element 53. In contrast, according to the modification illustrated in FIG. 23, the photoplethysmographic sensor 50 includes two light-receiving elements 53 and 54. Light output from the light-emitting element 51 and reflected from the measurement site is detected by the light-receiving element 53, and light output from the light-emitting element 52 and reflected from the measurement site is detected by the light-receiving element 54.

At the light-receiving surface of the light-receiving element 53, an optical filter is disposed that allows transmission of light output from the light-emitting element 51, and that blocks light at other wavelengths. At the light-receiving surface of the light-receiving element 54, an optical filter is disposed that allows transmission of light output from the light-emitting element 52, and that blocks light at other wavelengths.

According to some exemplary aspects, a range for the distance L1 from the light-receiving element 53 to the light-emitting element 51, and a range for the distance L2 from the light-receiving element 54 to the light-emitting element 52 are respectively the same as the range for the distance L1 and the range for the distance L2 in the physiological information measurement device (FIG. 12) according to the second exemplary embodiment.

As with this modification, the light-receiving elements 53 and 54 can be respectively disposed for the light-emitting elements 51 and 52. According to this modification, the two light-emitting elements 51 and 52 do not necessarily have to emit light at staggered timings but can be made to emit light at all times, or each of the two light-emitting elements 51 and 52 can be independently made to emit pulsed light at a predetermined frequency.

### Third Exemplary Embodiment

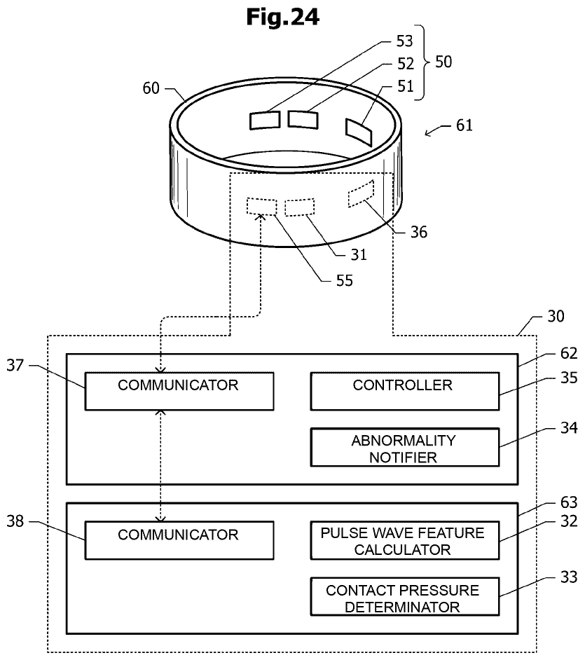

Reference is now made to FIG. 24 to describe a physiological information measurement device according to a third exemplary embodiment. In the following, structural features similar to those of the pulse wave signal processor and the physiological information measurement device according to the second exemplary embodiment described above with reference to FIGS. 12 to 22 are not described in further detail.

FIG. 24 illustrates, in perspective view, a portion of the physiological information measurement device according to the third exemplary embodiment, and illustrates, in block diagram, the pulse wave signal processor 30.

According to an exemplary aspect, two light-emitting elements 51 and 52, and a single light-receiving element 53 are mounted to the inner face of an attachment component 60 having an annular shape. In use, the attachment component 60 is attached on a user's finger. The attachment component 60 is available in a plurality of sizes depending on the thickness of the user's finger. Upon attaching the attachment component 60 on the finger, the light-emitting elements 51 and 52 output light toward the finger. The light-receiving element 53 is mounted at a position where light is incident on the light-receiving element 53 after being reflected from the interior of the finger. The attachment component 60 further incorporates the light emission controller 31, the pulse wave measurer 36, and a communicator 55. The light emission controller 31, the pulse wave measurer 36, and the communicator 55 can be implemented by a single integrated circuit. The attachment component 60, and the components mounted to the attachment component 60, including the light-emitting elements 51 and 52, the light-receiving element 53, the light emission controller 31, the pulse wave measurer 36, and the communicator 55, are herein referred to as “ring device 61.”

According to an exemplary aspect, the function of the pulse wave signal processor 30 is implemented by the ring device 61, a portable information terminal 62, and a server 63. Suitable examples of the information terminal 62 include a smartphone, a tablet terminal, and a notebook computer. The information terminal 62 includes a communicator 37, the controller 35, and the abnormality notifier 34. The server 63 includes a communicator 38, the pulse wave feature calculator 32, and the contact pressure determinator 33.

Moreover, data communication is performed between the communicator 55 of the ring device 61 and the communicator 37 of the information terminal 62, and between the communicator 37 of the information terminal 62 and the communicator 38 of the server 63. For the communication between the ring device 61 and the information terminal 62, for example, short-range wireless communication systems in compliance with various standards are used. For the communication between the information terminal 62 and the server 63, the Internet or other communication networks are used.

The light emission controller 31 is configured to control the emission of pulsed light by the light-emitting elements 51 and 52. The pulse wave measurer 36 is configured to read the signal from the light-receiving element 53. For example, the light emission controller 31 of the ring device 61 receives a command from the controller 35 of the information terminal 62 and causes the light-emitting elements 51 and 52 to emit light based on the received command. The pulse wave measurer 36 is configured to read out, from the light-receiving element 53, an intensity signal representing the intensity of received light, and to transmit the intensity signal to the server 63 via the information terminal 62. As with the pulse wave signal processor 30 (FIG. 12) according to the second exemplary embodiment, the pulse wave feature calculator 32 and the contact pressure determinator 33 of the server 63 calculate a pulse wave feature based on a pulse wave, and determine whether the contact pressure is abnormal.

The contact pressure determinator 33 is configured to transmit a determination result to the controller 35 of the information terminal 62. In response to receiving a determination result indicating that the contact pressure is abnormal, the controller 35 is configured to control the abnormality notifier 34, and notifies the user that the contact pressure is abnormal.

It should be appreciated that too large a ring size causes a gap to form between the photoplethysmographic sensor 50 and the skin, which makes it impossible to measure a pulse wave signal in a stable and accurate manner. Moreover, too small a ring size leads to an excessive contact pressure of the photoplethysmographic sensor 50 against the skin, which also decreases the reliability of the results of physiological information estimation. Accordingly, a method is now described for selecting the ring device 61 of an appropriate size from a plurality of different-sized ring devices 61.

First, the user puts on a plurality of ring devices 61 of different sizes in turn and activates the photoplethysmographic sensor 50 and the pulse wave signal processor 30. When the contact pressure is determined to be excessive for the ring device 61 of a certain size, another ring device 61 of a slightly larger size, for example, a size that is one or two levels larger than the size of the above-mentioned ring device 61 can be selected as the ring device 61 of the most appropriate size. In this way, when the contact pressure is determined to be excessive for a certain size, a size that is one or two levels larger than this size is selected. According to an exemplary aspect, selection of the ring device 61 of an inappropriate size can be avoided.

Reference is now made to advantages of the third exemplary embodiment.

As described herein, the finger is a suitable site for acquisition of a pulse wave using the photoplethysmographic sensor 50 due to its comparatively thin skin. Further, in the finger, the pathways for capillaries are less complex than those in the face or other sites. This means that a pulse wave feature acquired from the finger tends to be stable in value. This in turn improves the reliability of various physiological information determined from the pulse wave. A further advantage of the third exemplary embodiment is that, when the physiological information measurement device is used continuously or intermittently, it does not feel very awkward or uncomfortable for the user to wear the ring device 61 on the finger for an extended period of time.

An inappropriate size of the ring device 61 results in the inability to perform stable pulse wave measurement, or decreased reliability of the results of physiological information estimation. According to the third exemplary embodiment, the ring device 61 of an appropriate size can be selected from different-sized ring devices 61. According to an exemplary aspect, selecting the ring device 61 of an appropriate size can facilitate stable pulse wave measurement, and consequently improve the reliability of the results of physiological information estimation.

Reference is now made to modifications of the third exemplary embodiment.

According to an exemplary aspect, the function of the server 63 can be implemented in part or in whole by the information terminal 62. Moreover, the function of the information terminal 62 can be implemented in part by the ring device 61. For example, the function of the abnormality notifier 34 can be implemented by the ring device 61. For example, the attachment component 60 can incorporate a vibration generator, and when the contact pressure is determined to be abnormal, the vibration generator can be activated to vibrate the attachment component 60.

As for the contact pressure determinator 33, as described above with reference to the second exemplary embodiment, an estimation equation can be created in advance such that the degree of excessiveness of the contact pressure increases with decreasing pulse wave feature “ab-time” (FIG. 19), and the contact pressure determinator 33 can be configured to estimate the degree of excessiveness of the contact pressure from a measured value of the pulse wave feature “ab-time”, and providing notification of the degree of excessiveness. With the ring device 61 attached on a finger, bending the finger results in increased contact pressure, and straightening the finger results in decreased contact pressure. The abnormality notifier 34 instructs the user to bend or straighten the finger.

Based on how the degree of excessiveness of the contact pressure changes as the user straightens and bends the finger, the contact pressure determinator 33 determines whether the size of the ring device 61 is appropriate, and the abnormality notifier 34 notifies the user of the determination result. According to an exemplary aspect, the ring device 61 of an appropriate size accurately can be selected through a simple method.

Reference is now made to other modifications of the third exemplary embodiment.

Although the ring device 61 of the physiological information measurement device according to the third exemplary embodiment is attached to the finger, a device configured for attachment to a site other than the finger can be used instead of the ring device 61. For example, such a device can be a wearable device configured for attachment to the wrist, the neck, the face, the ear, or other sites. The physiological information measurement device does not necessarily have to be wearable but can be a device that the user uses to measure physiological information by pressing the photoplethysmographic sensor 50 against the finger as required. For example, the physiological information measurement device can be a portable device such as a smartphone, or can be a fixed installation device.

If the physiological information measurement device is of a portable or fixed installation type, the pulse wave signal processor 30 can, in response to the user bringing a measurement site into contact with the photoplethysmographic sensor 50, determine whether the contact pressure is within an appropriate range. If the photoplethysmographic sensor unit of the physiological information measurement device is a wearable device, the pulse wave signal processor 30 can, upon attachment of the wearable device, determine whether the contact pressure is within an appropriate range. For example, if the wearable device is a wristband- or wristwatch-type device to be attached to the wrist, pulse wave measurement can be performed upon attachment of the device, and if the contact pressure is determined to be abnormal, the abnormality notifier 34 can provide a notification to the user that instructs the user to loosen the belt of the device.

In general it is noted that the above-mentioned exemplary embodiments are for illustrative purposes only, and structural features described in different exemplary embodiments can be substituted for or combined with each another. The same or similar operational effects provided by the same or similar structural features according to a plurality of exemplary embodiments are not mentioned for each individual exemplary embodiment. Further, the above-mentioned exemplary embodiments are not intended to be limiting of the present disclosure. For example, various modifications, improvements, or combinations will be apparent to those skilled in the art.

### REFERENCE SIGNS LIST

30 pulse wave signal processor
31 light emission controller
32 pulse wave feature calculator
33 contact pressure determinator
34 abnormality notifier
35 controller
36 pulse wave measurer
37, 38 communicator
50 photoplethysmographic sensor
51, 52 light-emitting element
53, 54 light-receiving element
55 communicator
56 controller
60 attachment component
61 ring device
62 information terminal
63 server
70 body surface
71 epidermal region
72 arterioles
73 capillaries

What is claimed is:

1. A pulse wave signal processor comprising:
a pulse wave feature calculator configured to calculate a value of a pulse wave feature based on a steepness of a rise of a pulse wave that is measured with a photoplethysmographic sensor; and
a contact pressure determinator configured to, based on the value of the pulse wave feature, determine whether a contact pressure of the photoplethysmographic sensor pressing against a measurement site is within a range.

2. The pulse wave signal processor according to claim 1, wherein the value of the pulse wave feature is calculated based on a time difference between a first peak corresponding to an a-wave in an acceleration pulse wave and a second peak corresponding to a b-wave in the acceleration pulse wave, the acceleration pulse wave being based on a second-order derivative of the pulse wave.

3. The pulse wave signal processor according to claim 1, wherein the value of the pulse wave feature is calculated based on a width of a first peak within on beat of a velocity pulse wave, the velocity pulse wave being based on a first-order derivative of the pulse wave.

4. The pulse wave signal processor according to claim 1, wherein the value of the pulse wave feature is calculated based on a ratio between a peak value of an a-wave in an acceleration pulse wave, and an amplitude of the pulse wave, the acceleration pulse wave that is based on a second-order derivative of the pulse wave.

5. The pulse wave signal processor according to claim 1, wherein the value of the pulse wave feature is calculated based on a ratio between a first difference in an acceleration pulse wave and a second difference in the acceleration pulse wave, the first difference being a difference between a first peak value of an a-wave of the acceleration pulse wave and a second peak value of a b-wave of the acceleration pulse wave, and the second difference being a difference between the first peak value of the a-wave and a third peak value of a d-wave of the acceleration pulse wave, the acceleration pulse wave being based on a second-order derivative of the pulse wave.

6. The pulse wave signal processor according to claim 1, further comprising an abnormality notifier configured to, in response to the contact pressure determinator determining that the contact pressure is not within the range, notify that the contact pressure is abnormal.

7. A physiological information measurement device comprising:
a photoplethysmographic sensor configured to generate a pulse wave based on a measurement on a measurement site; and
a pulse wave signal processor that comprises:
a pulse wave feature calculator configured to calculate a value of a pulse wave feature based on a steepness of a rise of the pulse wave; and
a contact pressure determinator configured to, based on the value of the pulse wave feature, determine whether a contact pressure of the photoplethysmographic sensor pressing against the measurement site is within a range.

8. The physiological information measurement device according to claim 7, wherein the photoplethysmographic sensor includes:
at least a first light-emitting element configured to emit a first emitting light to the measurement site; and
at least a first light-receiving element configured to detect a first receiving light that is reflected from or transmitted through the measurement site in response to the first emitting light.

9. The physiological information measurement device according to claim 8, wherein the first light-emitting element is configured to output the first emitting light within a range of wavelengths from red to near-infrared.

10. The physiological information measurement device according to claim 9,
wherein the first light-receiving element is configured to detect the first receiving light that is reflected from the measurement site, and
wherein the first light-emitting element and the first light-receiving element are at a distance that is greater than or equal to 5 mm and is less than or equal to 20 mm from each other.

11. The physiological information measurement device according to claim 8,
wherein the photoplethysmographic sensor comprises the first light-emitting element configured to output the first emitting light at a first wavelength and a second light-emitting element configured to output a second emitting light at a second wavelength that is different from the first wavelength, and the photoplethysmographic sensor is configured to generate a first pulse wave associated with the first emitting light, and a second pulse wave associated with the second emitting light;
wherein the pulse wave feature calculator is configured to calculate a first value of the pulse wave feature based on a steepness of a rise of the first pulse wave, and a second value of the pulse wave feature based on a steepness of a rise of the second pulse wave, and
wherein the contact pressure determinator is configured to compare the first value of the pulse wave feature and the second value of the pulse wave feature to obtain a comparison result, and determine whether the contact pressure is within the range based on the comparison result.

12. The physiological information measurement device according to claim 11, wherein the second light-emitting element is configured to output the second emitting light at the second wavelength that is within a wavelength range from blue to yellow-green.

13. The physiological information measurement device according to claim 12,
wherein a first light-receiving element is configured to detect a first receiving light that is reflected from the measurement site, and
wherein the second light-emitting element and the first light-receiving element are at a distance of greater than or equal to 1 mm and less than or equal to 3 mm from each other.

14. The physiological information measurement device according to claim 8, further comprising:
an attachment component configured to be attached on a finger,
wherein the first light-emitting element and the first light-receiving element are mounted to the attachment component, and upon an attachment of the attachment component on the finger, the first light-emitting element outputs the first emitting light toward the finger, and the first receiving light is incident on the first light-receiving element after the first emitting light is reflected from an interior of the finger.

15. The physiological information measurement device according to claim 14, wherein the contact pressure determinator is configured to, based on the contact pressure, determine whether the attachment component has an appropriate size.

16. The physiological information measurement device according to claim 7, wherein the value of the pulse wave feature is calculated based on a time difference between a first peak corresponding to an a-wave in an acceleration pulse wave and a second peak corresponding to a b-wave in the acceleration pulse wave, the acceleration pulse wave being based on a second-order derivative of the pulse wave.

17. The physiological information measurement device according to claim 7, wherein the value of the pulse wave feature is calculated based on a width of a first peak within one beat of a velocity pulse wave, the velocity pulse wave being based on a first-order derivative of the pulse wave.

18. The physiological information measurement device according to claim 7, wherein the value of the pulse wave feature is calculated based on a ratio between a peak value of an a-wave in an acceleration pulse wave, and an amplitude of the pulse wave, the acceleration pulse wave being based on a second-order derivative of the pulse wave.

19. The physiological information measurement device according to claim 7, wherein the value of the pulse wave feature is calculated based on a ratio between a first difference in an acceleration pulse wave and a second difference in the acceleration pulse wave, the first difference being a difference between a first peak value of an a-wave of the acceleration pulse wave and a second peak value of a b-wave of the acceleration pulse wave, and the second difference being a difference between the first peak value of the a-wave and a third peak value of a d-wave of the acceleration pulse wave, the acceleration pulse wave being based on a second-order derivative of the pulse wave.

20. A method for determining a contact-pressure abnormality, the method comprising:
calculating a value of a pulse wave feature based on a steepness of a rise of a pulse wave, the pulse wave being measured with a photoplethysmographic sensor that is placed in contact with a measurement site; and
based on the value of the pulse wave feature, determining whether a contact pressure is within a range, the contact pressure being a pressure with which the photoplethysmographic sensor is pressed against the measurement site.
